(19) United States
(12) Patent Application Publication
(10) Pub. No.: US 2022/0087808 A1
(43) Pub. Date: Mar. 24, 2022

(54) Surgical Instruments for Insertion of Surgical Mesh and Related Methods

(71) Applicant: Winbock Medical LLC, Draper, UT (US)

(72) Inventors: Jeffrey Ben Winter, Vineyard, UT (US); Samuel Richard Zarbock, Draper, UT (US); Trey Scott Winter, Salt Lake City, UT (US)

(21) Appl. No.: 17/394,961

(22) Filed: Aug. 5, 2021

Related U.S. Application Data
(60) Provisional application No. 63/080,781, filed on Sep. 20, 2020.

(51) Int. Cl. A61F 2/00; A61B 17/34

(52) U.S. Cl. CPC A61F 2/0063; A61F 2230/0091; A61F 2002/0072; A61B 17/3468

(57) ABSTRACT

A surgical instrument for use in inserting a flexible sheet within a patient includes a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along an entire length of the tube between the proximal end face and the distal end face, the interior surface bounding a passage extending through the tube. A handle projects from the tube at or toward the proximal end.

### CROSS-REFERENCE TO RELATED APPLICATIONS

This application claims the benefit of U.S. Provisional Application No. 63/080,781, filed Sep. 20, 2020, which is incorporated herein by specific reference.

### BACKGROUND OF THE INVENTION

### 1. The Field of the Invention

The present invention relates to surgical instruments, devices, methods, and kits for delivering a flexible sheet, such as a surgical mesh, into a body cavity of a patient.

### 2. The Relevant Technology

Patients with hernias frequently undergo surgical repair using laparoscopic surgery. As part of this procedure, surgeons are required to insert a piece of sterilized mesh into the abdominal cavity of the patient through a trocar. A trocar includes a rigid cannula or tube having a sharpened distal tip which is placed through the abdominal wall. The cannula bounds a channel through which the mesh is passed for delivery into the abdominal cavity. However, because the channel is narrow, it can be difficult to efficiently pass the mesh through the trocar. Traditional approaches entail a surgeon manually folding or rolling the freely exposed mesh and then manipulating the mesh down the channel of the trocar. Lubrication can be used to help pass the mesh through the trocar.

Although passing the mesh through the trocar is typically successful, the process of both manipulating the mesh into a desired shape for fitting into the trocar and then advancing the mesh through the trocar is difficult, cumbersome, and time consuming. It can also result in unwanted tearing of the mesh. As such, the processing and positioning of the mesh can slow down the surgical procedure which lengthens operating time. In turn, lengthening the operating time increases the time that patients are under anesthesia, thereby increasing the risk to the patient and also increasing the cost of the operation.

Accordingly, what is needed in the art are medical devices and related methods that simplify and speed up the process of delivering a surgical mesh into an abdominal cavity of a patient so as to minimize patient risk, decrease cost, and/or limit tearing of the mesh. Although attempts have been made to develop such devices, such attempts have been expensive, complicated and have produced minimal improvement.

### SUMMARY OF THE DISCLOSURE

One independent aspect of the present disclosure includes a surgical instrument for use in inserting a flexible sheet within a patient, the surgical instrument comprising:
a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along an entire length of the tube between the proximal end face and the distal end face, the interior surface bounding a passage extending through the tube; and
a handle projecting from the tube at or toward the proximal end.

In one embodiment, the slot extends between the proximal end face and the distal end face is linear.

In one embodiment, the slot has a constant width at all locations along the slot between the proximal end face and the distal end face.

In one embodiment, the exterior surface of the tube comprises:
a top face and an opposing bottom face extending between the proximal end face and the distal end face; and
a first side face and an opposing second side face extending between the proximal end face and the distal end face and being disposed between the top face and the bottom face, wherein the slot extends along the top face between the proximal end face and the distal end face.

In one embodiment, the handle is coupled directly to and projects away from the bottom face.

In one embodiment, the handle is coupled directly to and projects away from the proximal end face.

In one embodiment, the handle is coupled to the proximal end of the tube and projects proximal of the proximal end face.

In one embodiment, the handle is curved.

In one embodiment, the handle has a U-shaped configuration.

One embodiment further includes an elongated first channel extending through the sidewall of the tube along the first side face so as to communicate with the passage.

In one embodiment, the first channel is spaced apart from the proximal end face and the distal end face of the tube.

One embodiment further includes an elongated second channel extending through the sidewall of the tube along the second side face so as to communicate with the passage.

In one embodiment, at least a majority of the length of the tube has a cylindrical configuration.

In one embodiment, the distal end of the tube is tapered so as to have a frustoconical configuration.

In one embodiment, the tube is comprised of a nonconductive, plastic material.

In one embodiment, the tube is comprised of acrylonitrile butadiene styrene.

In one embodiment, at least a portion of the exterior surface of the tube is textured.

In one embodiment, the textured exterior surface is a knurling pattern.

In one embodiment, there are multiple types of textured exteriors.

In one embodiment, the tube comprises two different tapered sections.

In one embodiment, the tube has a thicker rim on one side.

One embodiment further includes means for securing the flexible sheet to the distal end of tube.

In one embodiment, a notch extends through the sidewall at the distal end of the tube and passes through the distal end face.

In one embodiment, the notch is disposed opposite the slot.

In one embodiment, the notch is tapered.

One embodiment further includes a pointed catch projecting from the tube into or in alignment with the notch.

In one embodiment, a groove recessed is formed into the exterior surface of the tube at the distal end.

One embodiment further includes a cap removably secured to the distal end of the tube so as to at least partially cover the distal end face.

In one embodiment, the cap comprises:
tubular sleeve at least partially bounding a cavity and having a first end and opposing second end, opening being formed at the first end of the sleeve so as to communicate with the cavity, the distal end of the tube being disposed within the cavity; and
an end wall formed at the second end of the sleeve so as to at least partially bound the cavity.

The surgical instrument as recited in claim 29, further comprising an insert projecting from the end wall into the cavity of the sleeve, the insert being removably received within passage of the tube.

In one embodiment, an annular shoulder radially inwardly projecting from an interior surface of the sleeve into the cavity.

In one embodiment, a notch passing through the sleeve so as to communicate with the cavity.

In one embodiment, the notch also passes through a portion of the end wall.

In one embodiment, the portion of the notch passing through the end wall is tapered.

In one embodiment, a surgical system includes:
the surgical instrument; and
a flexible sheet being removably disposed within the passage of the tube.

In one embodiment, the flexible sheet comprises a surgical mesh.

In one embodiment, the flexible sheet within the passage of the tube is spirally wound.

A second independent aspect of the present disclosure includes a surgical instrument for use in inserting a flexible sheet within a patient, the surgical instrument comprising:
a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along at least a portion of a length of the tube between the proximal end face and the distal end face, the interior surface bounding a passage extending through the tube;

a notch extending through the sidewall at the distal end of the tube and passing through the distal end face; and
a handle projecting from the tube at or toward the proximal end.

In one embodiment, the notch is disposed opposite the slot.

In one embodiment, the notch is tapered.

In one embodiment, a pointed catch projects from the tube into or in alignment with the notch.

In one embodiment, a surgical system comprises:
the surgical instrument; and
a flexible sheet, a portion of the flexible sheet being secured within the notch.

In one embodiment, a portion of the flexible sheet is removably disposed within the passage of the tube.

A third independent aspect of the present disclosure includes a surgical instrument for use in inserting a flexible sheet within a patient, the surgical instrument, comprising:
a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along at least a portion of a length of the tube between the proximal end face and the distal end face, the interior surface bounding a passage extending through the tube;
a handle projecting from the tube at or toward the proximal end; and
a cap removably secured to the distal end of the tube so as to at least partially cover the distal end face.

The surgical instrument as recited in claim 44, wherein the cap comprises:
a tubular sleeve at least partially bounding a cavity and having a first end and an opposing second end, opening being formed at the first end of the sleeve so as to communicate with the cavity, the distal end of the tube being disposed within the cavity; and
an end wall formed at the second end of the sleeve so as to at least partially bound the cavity.

In one embodiment, an insert projects from the end wall into the cavity of the sleeve, the insert being removably received within passage of the tube.

In one embodiment, a notch passes through the sleeve so as to communicate with the cavity.

In one embodiment, the notch also passes through a portion of the end wall.

In one embodiment, the portion of the notch passing through the end wall is tapered.

In one embodiment, a surgical system comprising:
the surgical instrument; and
a flexible sheet, a portion of the flexible sheet being secured to the cap or being secured between the cap and the tube.

In one embodiment, a portion of the flexible sheet is removably disposed within the channel of the tube.

In a fourth independent aspect of the present disclosure, a method for deploying a sheet of surgical mesh includes:
securing a first portion of a sheet of surgical mesh to a distal end of a tube of a surgical instrument, the tube having a sidewall with an interior surface and an opposing exterior surface that extend between a proximal end and the opposing distal end, the proximal end terminating at a proximal end face and the distal end terminating at a distal end face, a slot passing through the sidewall of the tube between the interior surface and the exterior surface and extending along at least a portion of a length of the tube between the proximal end face and the distal end face, the interior surface bounding a passage extending through the tube; and
passing a second portion of the sheet through the slot of the tube and into the passage.

In one embodiment, the step of securing the first portion of the sheet comprises securing the sheet within a notch extending through the sidewall at the distal end of the tube and passing through the distal end face.

In one embodiment, the step of securing the first portion of the sheet comprises capturing the first portion of the sheet between the distal end of the tube and a cap removably secured to the distal end of the tube so as to at least partially cover the distal end face.

In one embodiment, the step of securing the first portion of the sheet comprises securing the first portion of the sheet to a cap removably secured to the distal end of the tube so as to at least partially cover the distal end face.

In one embodiment, after the step of passing a second portion of the sheet through the slot, twisting the sheet so that at least a portion of the sheet is spirally wound within the passage of the tube.

In one embodiment, the step of twisting the sheet comprises grasping a portion of the sheets with forceps and manipulating the forceps to twist the sheet.

One embodiment further includes releasing the first portion of a sheet from the distal end of a tube.

One embodiment further includes:
positioning the distal end of the tube within a first end of a channel of a trocar; and
advancing a pushing tool through the passage of the tube and through the of the trocar so that the pushing tool pushes the sheet into and through the channel of the trocar.

In one embodiment, the pushing tool comprises a laparoscopic grasper.

A fifth independent aspect of the present disclosure includes a surgical instrument for use in inserting a flexible sheet within a patient, the surgical instrument comprising:
a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along at least a portion of a length of the tube between the proximal end face and the distal end face, the interior surface bounding a passage extending through the tube; and
a handle projecting from the tube at or toward the proximal end.

It is understood that each of the above alternative features associated with each of the independent aspects and all other features disclosed throughout the specification and claims can be combined with and/or mixed and matched with each of the independent aspects.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1A is a top perspective view of a surgical instrument for use in inserting a flexible sheet within a cavity of a patient;

FIG. 1B is a bottom perspective view of the surgical instrument shown in FIG. 1;

FIG. 1C is a top plan view of the surgical instrument shown in FIG. 1;

FIG. 1D is an elevated side view of the surgical instrument shown in FIG. 1;

FIG. 2 is a top plan view of flexible sheet for deployment by the surgical instrument shown in FIG. 1;

FIG. 3 is a perspective view of the flexible sheet being secured at the distal end of the surgical instrument;

FIG. 4 is a perspective view of the flexible sheet shown in FIG. 3 being inserting along the length of a tube of the surgical instrument;

FIG. 5 is a perspective view of the flexible sheet being coiled within surgical instrument shown in FIG. 4;

FIG. 6 is a perspective view of the surgical instrument with flexible sheet being aligned for use with a trocar and pushing tool;

FIG. 7 is a perspective view of the assembly in FIG. 6 in use;

FIG. 8A is a top perspective view of an alternative embodiment of the surgical instrument shown in FIG. 1A with notch formed at the distal end of the tube;

FIG. 8B is a bottom perspective view of the surgical instrument shown in FIG. 8A;

FIG. 9 is a top perspective view of an alternative embodiment of the surgical instrument shown in FIG. 8A with a catch projecting into the notch;

FIG. 10 is a top perspective view of an alternative embodiment of the surgical instrument shown in FIG. 8A with the notch being tapered;

FIG. 11A is an exploded elevated side view of a surgical instrument comprising an insertion device with a cap;

FIG. 11B is an elevated side view of a surgical instrument shown in FIG. 11A in an assembled state;

FIG. 12 is an enlarged perspective view of the cap shown in FIG. 11A;

FIG. 13 is a perspective view of an alternative embodiment of the cap shown in FIG. 11A with a central projection;

FIG. 14 is a perspective view of an alternative embodiment of the cap shown in FIG. 11A with slots formed thereon;

FIG. 15 is a perspective view of an alternative embodiment of the cap shown in FIG. 11A with a slot and end notch formed thereon;

FIG. 16 is an elevated side view of a surgical instrument comprising an insertion device with a clamp;

FIG. 17 is a perspective view of one embodiment of the clamp shown in FIG. 16 in the form of a slide clamp;

FIG. 18 is a perspective view of one embodiment of the clamp shown in FIG. 16 in the form of a hinge clamp;

FIG. 19 is a perspective view of an alternative embodiment of the surgical instrument shown in FIG. 1A with a portion of the slot covered at the distal end;

FIG. 20 is a perspective view of an alternative embodiment of the surgical instrument shown in FIG. 1A with a portion of the slot covered at the proximal end;

FIG. 21 is a perspective view of an alternative embodiment of the surgical instrument shown in FIG. 1A having opposing secondary slide slots; and

FIG. 22 is a perspective view of the surgical instrument shown in FIG. 21 having flexible sheet extending through the opposing secondary slide slots.

### DETAILED DESCRIPTION OF THE PREFERRED EMBODIMENTS

Before describing the present disclosure in detail, it is to be understood that this disclosure is not limited to parameters of the particularly exemplified systems, methods, apparatus, products, processes, compositions, and/or kits, which may, of course, vary. It is also to be understood that the terminology used herein is only for the purpose of describing particular embodiments of the present disclosure, and is not necessarily intended to limit the scope of the disclosure in any particular manner. Thus, while the present disclosure will be described in detail with reference to specific embodiments, features, aspects, configurations, etc., the descriptions are illustrative and are not to be construed as limiting the scope of the claimed invention. Various modifications can be made to the illustrated embodiments, features, aspects, configurations, etc. without departing from the spirit and scope of the invention as defined by the claims. Thus, while various aspects and embodiments have been disclosed herein, other aspects and embodiments are contemplated.

Unless defined otherwise, all technical and scientific terms used herein have the same meaning as commonly understood by one of ordinary skill in the art to which the present disclosure pertains. While a number of methods and materials similar or equivalent to those described herein can be used in the practice of the present disclosure, only certain exemplary materials and methods are described herein.

Various aspects of the present disclosure, including devices, sys, methods, etc., may be illustrated with reference to one or more exemplary embodiments or implementations. As used herein, the terms “alternative embodiment” and/or “exemplary implementation” means “serving as an example, instance, or illustration,” and should not necessarily be construed as preferred or advantageous over other embodiments or implementations disclosed herein. In addition, reference to one or more embodiments is intended to provide illustrative examples without limiting the scope of the invention, which is indicated by the appended claims rather than by the following description.

It will be noted that, as used in this specification and the appended claims, the singular forms “a,” “an” and “the” include plural referents unless the content clearly dictates otherwise. Thus, for example, reference to an “insert” includes one, two, or more inserts. As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” variants thereof (e.g., “includes,” “has,” and “involves,” “contains,” etc.), and similar terms as used herein, including the claims, shall be inclusive and/or open-ended, shall have the same meaning as the word “comprising” and variants thereof (e.g., “comprise” and “comprises”), and do not exclude additional, un-recited elements or method steps, illustratively.

Various aspects of the present disclosure can be illustrated by describing components that are coupled, attached, connected, and/or joined together. As used herein, the terms “coupled”, “attached”, “connected,” and/or “joined” are used to indicate either a direct connection between two components or, where appropriate, an indirect connection to one another through intervening or intermediate components. In contrast, when a component is referred to as being “directly coupled”, “directly attached”, “directly connected,” and/or “directly joined” to another component, no intervening elements are present or contemplated. Thus, as used herein, the terms “connection,” “connected,” and the like do not necessarily imply direct contact between the two or more elements. In addition, components that are coupled, attached, connected, and/or joined together are not necessarily (reversibly or permanently) secured to one another.

As used herein, directional and/or arbitrary terms, such as “top,” “bottom,” “front,” “back,” “left,” “right,” “up,” “down,” “upper,” “lower,” “inner,” “outer,” “internal,” “external,” “interior,” “exterior,” “proximal,” “distal” and the like can be used solely to indicate relative directions and/or orientations and may not otherwise be intended to limit the scope of the disclosure, including the specification, invention, and/or claims.

Where possible, like numbering of elements have been used in various figures. Furthermore, alternative configurations of a particular element may each include separate letters appended to the element number. Accordingly, an appended letter can be used to designate an alternative design, structure, function, implementation, and/or embodiment of an element or feature without an appended letter. Similarly, multiple instances of an element and or sub-elements of a parent element may each include separate letters appended to the element number. In each case, the element label may be used without an appended letter to generally refer to instances of the element or any one of the alternative elements. Element labels including an appended letter can be used to refer to a specific instance of the element or to distinguish or draw attention to multiple uses of the element. However, element labels including an appended letter are not meant to be limited to the specific and/or particular embodiment(s) in which they are illustrated. In other words, reference to a specific feature in relation to one embodiment should not be construed as being limited to applications only within said embodiment.

It will also be appreciated that where a range of values (e.g., less than, greater than, at least, and/or up to a certain value, and/or between two recited values) is disclosed or recited, any specific value or range of values falling within the disclosed range of values is likewise disclosed and contemplated herein. Thus, disclosure of an illustrative measurement or distance less than or equal to about 10 units or between 0 and 10 units includes, illustratively, a specific disclosure of: (i) a measurement of 9 units, 5 units, 1 units, or any other value between 0 and 10 units, including 0 units and/or 10 units; and/or (ii) a measurement between 9 units and 1 units, between 8 units and 2 units, between 6 units and 4 units, and/or any other range of values between 0 and 10 units.

It is also noted that systems, methods, apparatus, devices, products, processes, compositions, and/or kits, etc., according to certain embodiments of the present disclosure may include, incorporate, or otherwise comprise properties, features, aspects, steps, components, members, and/or elements described in other embodiments disclosed and/or described herein. Thus, reference to a specific feature, aspect, steps, component, member, element, etc. in relation to one embodiment should not be construed as being limited to applications only within said embodiment. In addition, reference to a specific benefit, advantage, problem, solution, method of use, etc. in relation to one embodiment should not be construed as being limited to applications only within said embodiment.

The headings used herein are for organizational purposes only and are not meant to be used to limit the scope of the description or the claims. To facilitate understanding, like reference numerals have been used, where possible, to designate like elements common to the figures.

The present disclosure is directed to surgical instruments, devices, methods, and kits for delivering a flexible sheet, such as a surgical mesh, into a body cavity of a patient, such as an abdominal cavity. Although the surgical instruments, devices, methods, and kits will commonly be used in a laparoscopic surgical procedure for the repair of a hernia, the surgical instruments, devices, methods, and kits can also be used in other surgical procedures, such as other laparoscopic surgical procedures, where it is desired to insert a flexible sheet into a body cavity of a patient.

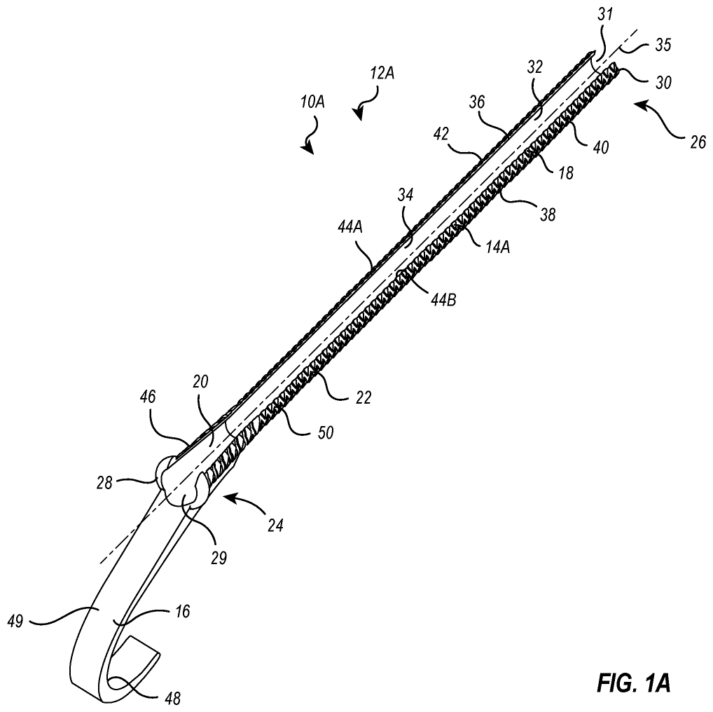

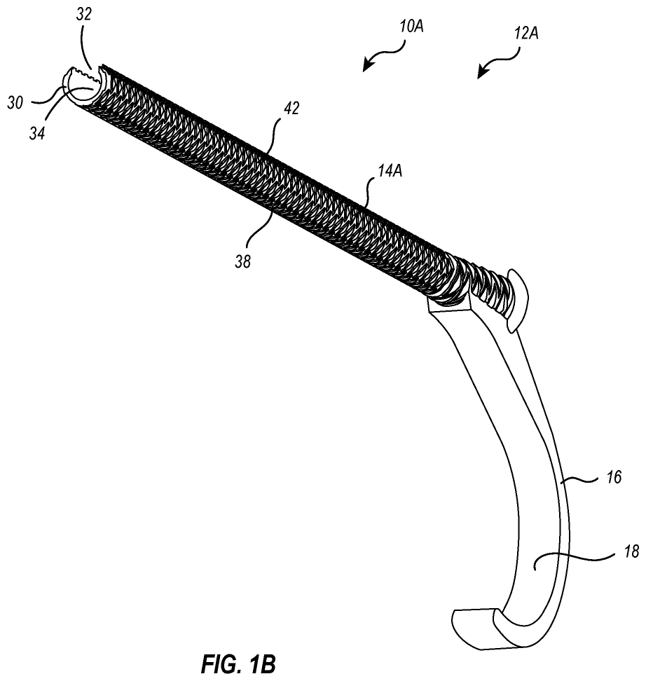

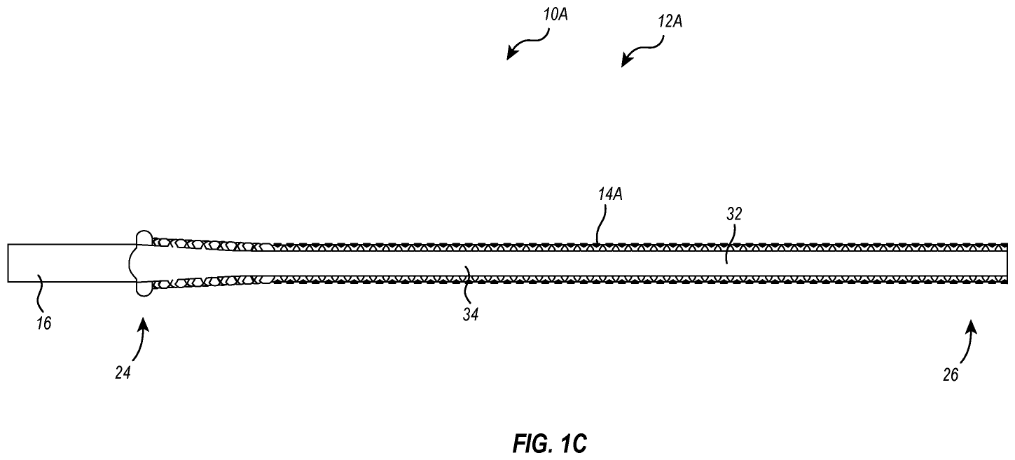

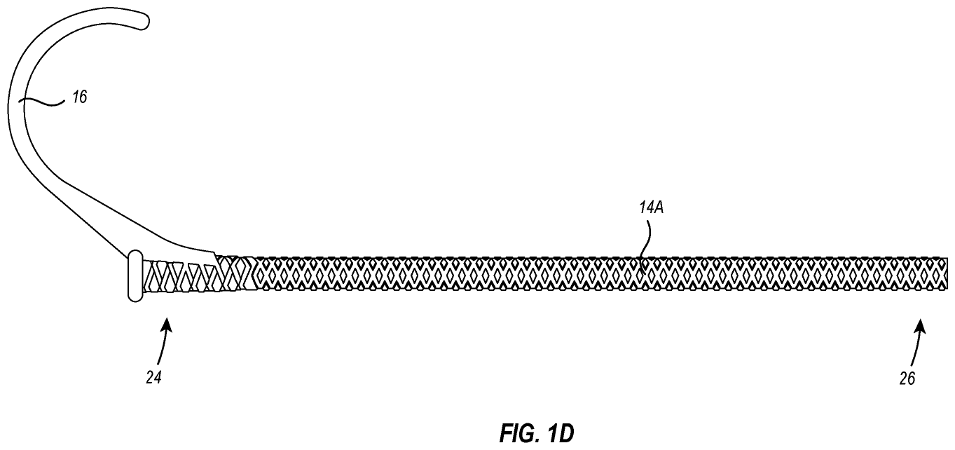

Depicted in FIGS. 1A-1D is a first embodiment of a surgical instrument 10A for use in inserting a flexible sheet within a body cavity of a patient. In this embodiment, surgical instrument 10A comprises an insertion device 12A that includes an elongated tube 14A having a handle 16 projecting therefrom. As discussed below in further detail, surgical instrument 10A can further include a cap that removably couples to insertion device 12A. Tube 14A has a sidewall 18 with an interior surface 20 and an opposing exterior surface 22 that extend between a proximal end 24 and an opposing distal end 26. Proximal end 24 terminates at a proximal end face 28 while distal end 26 terminates at a distal end face 30. Interior surface 20 bounds a passage 34 extending through tube 14A between proximal end 24 and distal end 26 and more specifically between proximal end face 28 and distal end face 30. Passage 34 has a central longitudinal axis 35 that extends along the length thereof.

A slot 32 passes through sidewall 18 of tube 14A between interior surface 20 and exterior surface 22 so as to communicate with passage 34. Slot 32 extends along an entire length of tube 14A between proximal end face 28 and distal end face 30. More specifically, exterior surface 22 of tube 14A includes a top face 36 and an opposing bottom face 38 that extend between proximal end face 28 and distal end face 30. Exterior surface 22 also includes a first side face 40 and an opposing second side face 42 that extend between proximal end face 28 and distal end face 30 and that are disposed between the top face 36 and bottom face 38. Slot 32 extends along top face 36 between proximal end face 28 and distal end face 30 and is bounded between opposing edges 44A and 44B of tube 14A extending between proximal end face 28 and distal end face 30.

Tube 14A is typically linear. For example, sidewall 18 and/or passage 34 and/or central longitudinal axis 35 typically extend linearly along the length of tube 14A or along at least a majority of the length of tube 14A. The length of tube 14A extending between proximal end face 28 and distal end face 30 is typically in a range between 10 cm and 30 cm with between 10 cm and 25 cm or between 10 cm and 20 cm being more common. Other dimensions can also be used based on application. Passage 34 has an inner diameter that is typically in a range between 0.3 cm and 2 cm with between 0.3 cm and 1.5 cm or between 0.5 cm and 1.5 cm being more common. Other dimensions can also be used based on application. The foregoing inner diameter can be a maximum diameter or a minimum diameter. In one embodiment, tube 14A or at least a majority of the length of tube 14A can have a cylindrical configuration. Thus, in some embodiments, exterior surface 22 and/or interior surface 20 or at least a majority of the lengths thereof can have a cylindrical configuration. However, tube 14A need not be cylindrical. For example, interior surface 20 and/or exterior surface 22 can have a transverse cross section normal to longitudinal axis 33 that is circular, oval, polygonal, rectangular or have other configurations. Furthermore, interior surface 20 and exterior surface 22 can have the same cross-sectional configurations or different cross sectional configurations.

In one embodiment, slot 32 has a width extending between edges 44A and 44B that is in a range between 0.2 cm and 1.5 cm with between 0.2 cm and 1 cm or between 0.3 cm and 0.8 cm being more common. Other dimensions can also be used based on application. As will be discussed below in more detail, if slot 32 is too large the sheet that is deployed by insertion device 12A can laterally escape out through slot 32 whereas if slot 32 is too narrow, it can be difficult to insert the sheet within passage 34. The width of slot 32 can be constant along the length of tube 14A or along at least a majority of the length of tube 14A. Furthermore, slot 32 or at least a majority of the length of slot 32 can extend linearly along the length of tube 14A. As such, edges 44A and 44B can be disposed in parallel alignment along the length of tube 14A or along at least a majority of the length of tube 14A. As used throughout the specification and appended claims, the term “majority” broadly includes at least 50%, 60%, 70%, 80%, 90%, or 95% with respect to the identified frame of reference. For example, stating that at least a majority of the length of slot 32 can extend linearly along the length of tube 14A discloses that at least 50%, 60%, 70%, 80%, 90%, or 95% of the length of slot 32 can extend linearly along the length of tube 14A.

In the depicted embodiment, a section 46 of tube 14A disposed at proximal end 24 and extending to proximal end face 28 radially outwardly flares towards proximal end face 28. In turn, the corresponding section of passage 34 and slot 32 also outwardly flare. As will be discussed below in greater detail, this flaring of tube 14A can assist in guiding related instruments into passage 34 at proximal end 24. However, in alternative embodiments, the flaring of tube 14A at section 46 can be eliminated.

Handle 16 projects from tube 14A at proximal end 24. In one embodiment, handle 16 is integrally formed as a single unity member with tube 14A. For example, tube 14A and handle 16 can be molded together. In alternative embodiments, handle 16 can connected to tube 14A such as by welding, adhesive, crimp fit, fastener, or the like. In the depicted embodiment, handle 16 projects from bottom face 38 of tube 14A so as to be opposite of slot 32 and also projects proximally directly from proximal end face 28. Handle 16 projects from tube 14A so that at least a portion of handle 16 projects proximal of proximal end face 28. Handle 16 is shown having a U-shaped configuration with a concave curved inside face 48 and a convex curved outside face 49. Handle 16 is ergonomically designed so that during use, handle 16 can be comfortably pinched between an operator's index finger placed on inside face 48 and an operator's thumb that can be placed on outside face 49 adjacent to proximal end face 28 of tube 14A and/or directly against proximal end face 28. This configuring and positioning of handle 16 makes it easy to grasp, manipulate and operate insertion device 12A. Furthermore, this positioning of handle 16 helps to eliminate or at least minimize interference by handle 16 during operation. In other embodiments, handle 16 can be moved a distance distally so as not to project proximal of proximal end face 28. Furthermore, although there are unique ergonomic benefits associated with the configuration of handle 16, in alternative embodiments, handle 16 could be in the form of a closed loop or have other configurations.

In the depicted embodiment, a texture 50 is formed on exterior surface 22 of tube 14A. Texture 50 can comprise knurling, ribs or any other form of projects that assists in griping tube 14A as needed. Texture 50 can be constant along tube 14A or can change at different locations. For example, texture 50 at tapered section 46 of tube 14A if different from texture 50 along the remaining length of tube 14A. In still other embodiments, as disclosed later, texture 50 can be eliminated.

Surgical instrument 10A/insertion device 12A can be made from a metal, such as stainless steel. In this embodiment, insertion device 12A can be reused by sterilization between uses. More commonly, however, surgical instrument 10A/insertion device 12A is formed from a polymeric material, such as a non-conductive, plastic material. When made from a relatively inexpensive material, insertion device 12A can be disposed of after a single use. In one embodiment, surgical instrument 10A/insertion device 12A and/or tube 14A are formed from acrylonitrile butadiene styrene (ABS) or polypropylene. It can be useful to use materials that can be sterilized by steam sterilization and/or irradiation. It can also be desirable to use materials that are sturdy, low cost, produce a smooth surface that enables easy sliding of the surgical mesh and/or is recyclable. Other materials can also be used.

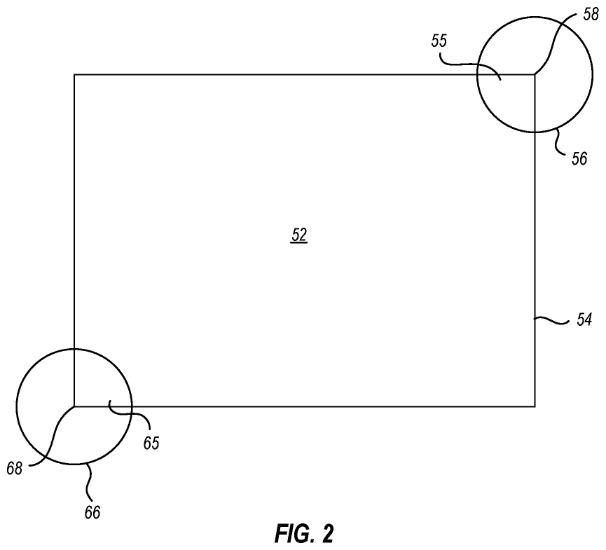

Turing to FIG. 2, surgical instrument 10A/insertion device 12A is used for deploying a flexible sheet 52 into a cavity of a patient, such as an abominable cavity. In one embodiment, sheet 52 comprises a sheet of surgical mesh used for hernia repair. Sheet 52 is commonly made from a polymeric material and can be porous. Sheet 52 is sufficiently flexible that it can be coiled within passage 34 of insertion device 12A and then removed and uncoiled without plastic deformation. It is appreciated that sheet 52 can comprise any type of surgical mesh or other types of sheets that are intended for implanting within a cavity of a patient.

During use, sheet 52 can pre-sized for an intended application or can be cut by the surgeon to a desired configuration or size. As shown in FIG. 2, sheet 52 has a perimeter edge 54 that is square or rectangular when sheet 52 is laid flat. However, sheet 52 can have any desired configuration such as circular, oval, polygonal, or have an irregular configuration. The configuration and size of sheet 52 depends upon the intended application.

Although sheet 52 can be deployed by insertion device 12A using a variety of different techniques, in one general method of use, a first portion of sheet 52 is secured at distal end 26 of tube 14A. While the first portion is being held secured at distal end 26, a further portion of sheet 52 projecting proximal of the first portion is coiled and positioned within passage 34 of insertion device 12A. The first portion of sheet 52 is then released from distal end 26. Distal end 26 of tube 14A is then aligned with a proximal end of a trocar having a channel that communicates with a body cavity of a patient. A push tool is then passed down through passage 34 of tube 14A so as to push the coiled sheet 52 through passage 34, through the channel of the trocar and into the body cavity of the patient.

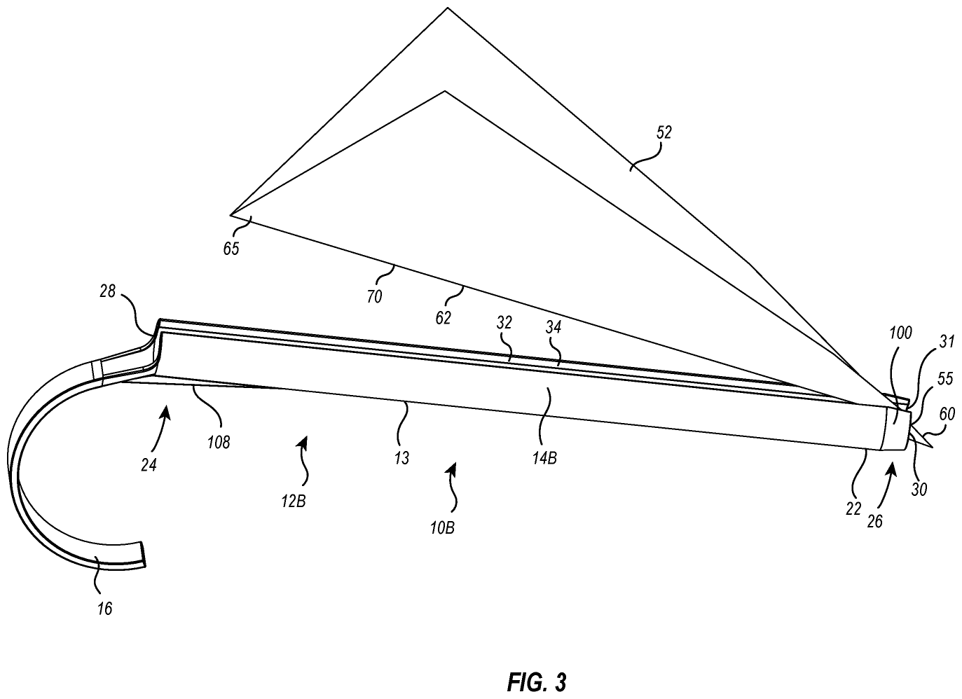

Depicted in FIGS. 3-7 is a more specific example of how an inventive insertion device can be used to deploy sheet 52 into a body cavity of a patient. Although insertion device 12A and all of the other insertion devices 12 disclosed herein can be used in the example illustrated in FIGS. 3-7, FIGS. 3-7 shows the use of an alternative embodiment of a surgical instrument 10B comprising an insertion device 12B. Insertion device 12B comprises a tube 14B having handle 16 projecting therefrom. Insertion device 12B is simply an example of how insertion devise 12A can be modified and like elements between insertion device 12A and 12B are identified by like reference characters. Specifically, except as noted below, insertion device 12A and 12B are the same and all of the prior discussion of insertion device 12A, including all discussions of elements, alternatives, materials, sizes, uses and the like are also applicable to insertion device 12B. Likewise, unless otherwise noted, all of the above discussion of elements, alternatives, materials, sizes, uses and the like for insertion device 12A are also applicable to all other insertion device 12 disclosed herein. Tube 14B differs from tube 14A in that distal end 26 includes a nose 100 extending to distal end face 30 that is tapered so as to have a frustoconical configuration. As will be discussed below in more detail, the formation of tapered nose 100 can help to direct nose 100 into the channel of a trocar and can prevent tube 14B from advancing too far down the channel of the trocar. As also discussed below, the tapering of nose 100 can assist with coupling a cap to distal end 26. As with tube 14A, however, tapered nose 100 is not required.

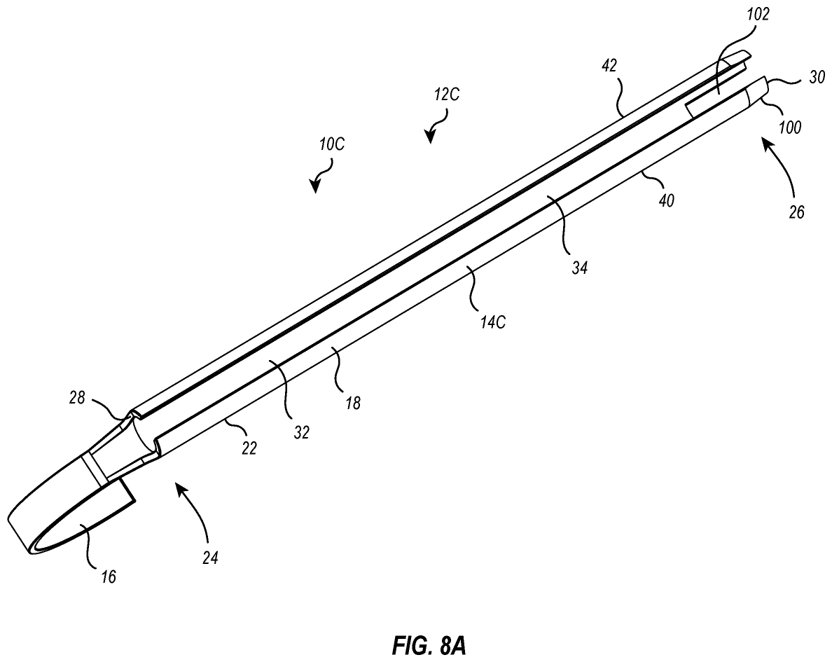

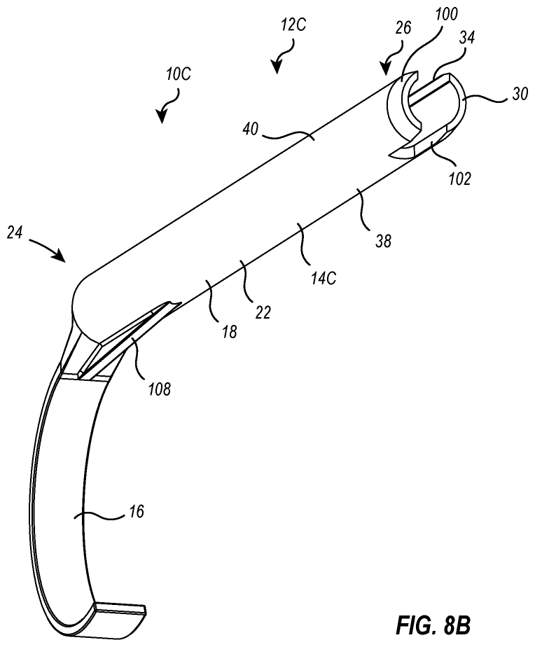

Insertion device 12B also has other modifications relative to insertion device 12A. For example, tube 14B does not include texture 50 (FIG. 1) on exterior surface 22. Furthermore, handle 16 does not project from bottom face 38 of tube 14B but rather only projects directly from proximal end face 28. However, an optional reinforcing brace 108 (FIG. 8B) extends between tube 14B and handle 16. Finally, it is noted that proximal end 24 of sidewall 18 of tube 14B does not taper but maintains a constant diameter, i.e., tapered section 46 (FIG. 1) has been eliminated.

With reference to FIG. 2, initially a surgeon or other operator grasps a first portion 55 of sheet 52. First portion 55 comprises the section of sheet 52 within circle 56 and typically includes a section of perimeter edge 54. In this embodiment, first portion 55 includes a corner 58 of sheet 52 but this is not required. As will be discussed below in more detail, sheet 52 also includes a second portion 65 that is disposed at an end of sheet 52 that is typically opposite of first portion 55. Second portion 65 comprises the section of sheet 52 within circle 66 and typically includes a section of perimeter edge 54. Again, in this embodiment, second portion 65 includes a corner 68 of sheet 52 (corner 68 being opposite of corner 58) but it is not required.

With reference to FIG. 3, the surgeon positions first portion 55 of sheet 52 within distal end 26 of passage 34 so that a terminal section 60 of first portion 55 projects out of passage 34 through distal opening 31. First portion 55 is then secured to distal end 26 of tube 14B. As will be discussed below in greater detail, this securing of first portion 55 to distal end 26 can be accomplished in a variety of different ways. However, in the depicted embodiment, this can be accomplished by the surgeon folding over terminal section 60 and using the surgeon's finger to press terminal section 60 against distal end 26 of tube 14A, e.g., pressing terminal section 60 against distal end face 30 and/or against exterior surface 22 at distal end 26.

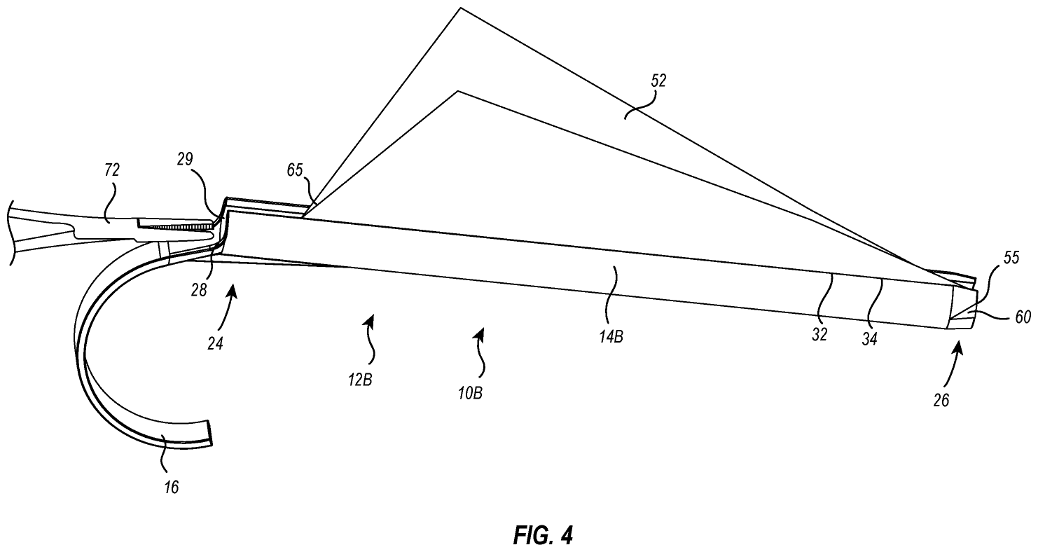

Next, in one embodiment, an elongated section 62 of sheet 52 projecting proximal of first portion 55 is advanced through slot 32 and into passage 34. Elongated section 62 can include a linear section of sheet 52 extending between first portion 55 and second portion 65. For example, sheet 52 can be folded so that elongated section 62 includes a fold edge or crease 70 extending between first portion 55 and second portion 65. The surgeon can then slide fold edge or crease 70/elongated section 62 through slot 32 and into passage 34, as shown in FIG. 4. Alternatively, or in combination, the surgeon can position sheet 52 on top of tube 14B. A tool can then be used to press elongated section 62 through slot 32 and into passage 34.

Either prior to or after positioning elongated section 62 of sheet 52 into passage 34, a grasping tool 72 is used to grasp second portion 65 of sheet 52. Grasping tool 72 can comprise forceps or other surgical graspers that enable a surgeon to both grasp and twist sheet 52. For example, the surgeon can first use grasping tool 72 to grasp second portion 65 of sheet 52 outside of passage 34 and then use grasping tool 72 to direct elongated section 62 through slot 32 and into passage 34. Alternatively, elongated section 62 can first be manually passed through slot 32 and into passage 34. Grasping tool 72 can then be used to grasp second portion 65, such as by using grasping tool 72 to reach into passage 34 through proximal opening 29. Sheet 52 and tube 14B are typically sized so that with elongated section 62 within passage 34, second portion 65 is also disposed within passage 34. That is, when sheet 52 is pulled taught between first portion 55 and second portion 65, second portion 65 is typically disposed within passage 34, i.e., no portion of sheet 52 projects out of passage 34 through proximal opening 29. This helps to ensure that sheet 52 does not bind against proximal end face 28 during dispensing of sheet 52 out of tube 14B, as will be discussed below. Accordingly, grasping tool 72 can be sized, in one embodiment, to pass through proximal opening 29 and grasp second portion 65 within passage 34.

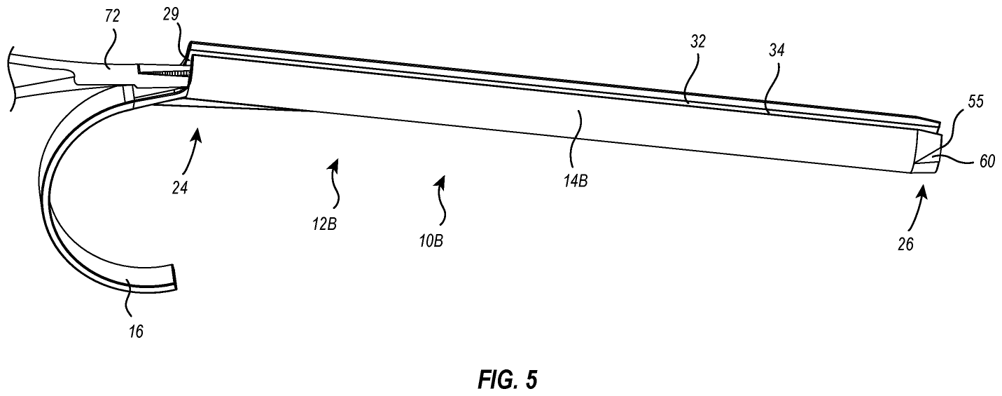

With elongated section 62 (FIG. 3) disposed within passage 34 and grasping tool 72 grasping second portion 65, sections of sheet 52 still remain outside of tube 14B, as shown in FIG. 4. In this position, grasping tool 72 is rotated (while first portion 55 remains secured at distal end 26) so that sheet 52 is progressively coiled within passage 34. As sheet 52 is coiled, the sections of sheet 52 outside of tube 14B are progressively drawn into passage 34 through slot 32. Grasping tool 72 is continually rotated until all or substantially all of sheet 52 between first portion 55 and second portion 65 is coiled and disposed within passage 34. Any portion of sheet 52 that projects out of passage 34 through slot 32 can simply be pressed through slot 32 and into passage 34, as shown in FIG. 5.

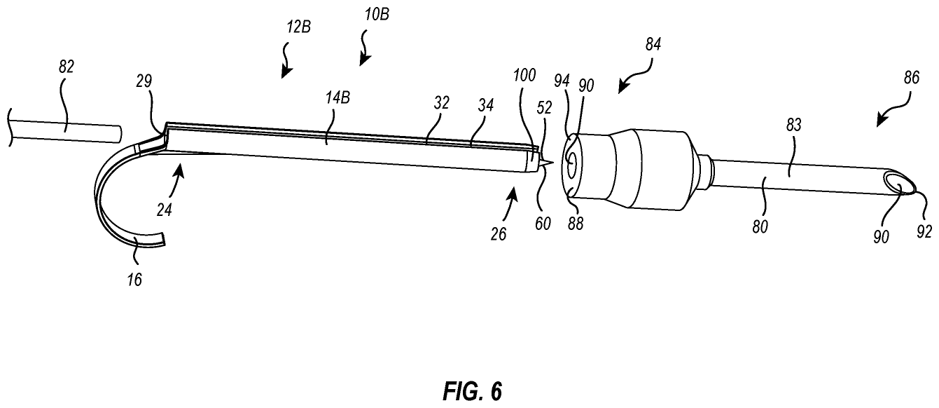

With sheet 52 coiled within passage 34, grasping tool 72 can be released from sheet 52 at proximal end 24 and removed from passage 34. In addition, first portion 55 of sheet 52 can be released at distal end 26. Sidewall 18 of tube 14B retains sheet 52 coiled within passage 34, as shown in 5. Where terminal section 60 was folded over to facilitate securing, terminal section 60 can now be straightened so as to be aligned with passage 34, as shown in FIG. 6.

With continued reference to FIG. 6, surgical instrument 10B/insertion device 12B is now used in concert with a trocar 80 and a pushing tool 82. Trocar 80 includes an elongated, tubular, cannula 83 that extends between a proximal end 84 and an opposing distal end 86. Cannula 83 has an interior surface 88 that bound a channel 90 extending through trocar 80 between proximal end 84 and distal end 86. Distal end 86 typically terminates at a sharpened tip 92 to facilitate puncturing through tissue. Channel 90 of trocar 80 commonly outwardly flares at proximal end 84 to a proximal end face 94. The flaring of channel 90 at proximal end 84 helps to facilitate guiding of elements, such as insertion device 12B, into channel 90. It is appreciated the trocars are well known in the art and come in a variety of different sizes and configurations which can be used in the present invention. In some embodiments, trocars can comprise two or more parts that are assembled together during use.

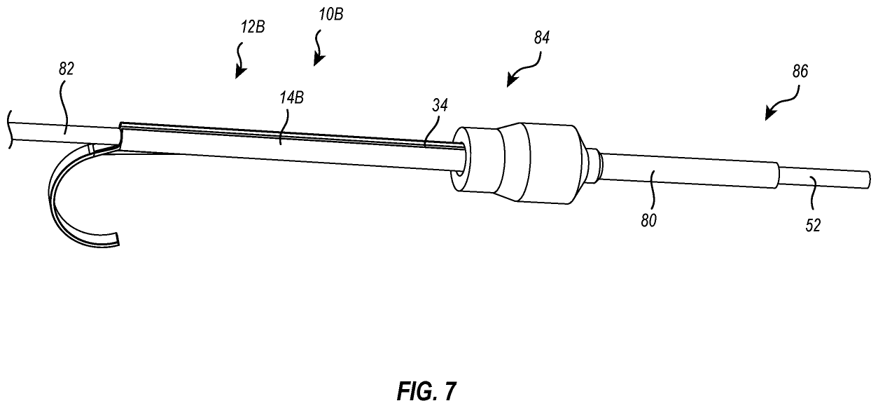

Continuing with the current method, distal end 86 of trocar 80 is passed through tissue of a patient so that distal end 86 communicates with a cavity of a patient, such as an abdominal cavity. When it is desired to deploy coiled sheet 52 into the cavity, distal end 26 of surgical instrument 10B/insertion device 12B is advanced into proximal end 84 of channel 90 until distal end 26 of tube 14B butts against interior surface 88 of trocar 80. The tapering of nose 100 can help guide distal end 26 into channel 90 and also prevents distal end 26 from pass through or too far into channel 90. In this position, as shown in FIG. 7, passage 34 of tube 14B is aligned with channel 90 of trocar 80. Likewise, coiled sheet 52 within passage 34 is aligned with channel 90 of trocar 80. While holding surgical instrument 10B/insertion device 12B with one hand using handle 16, the surgeon than uses the other hand to advance pushing tool 82 into proximal opening 29 of tube 14B, along passage 34 and into channel 90 of trocar 80. Pushing tool 82 is sized and configured so that as pushing tool 82 advances within passage 34 and channel 90, pushing tool 82 pushes coiled sheet 52 through passage 34 and channel 90.

Pushing tool 82 is advanced until coiled sheet 52 passes out through distal end 86 of trocar 80 and into the body cavity, as shown in FIG. 7. Pushing tool 82 can comprise any elongated member that can be sterilized and passed through passage 34 and channel 90 so as to advance coiled sheet 52 therethrough. In one typical embodiment, pushing tool 82 comprise a laparoscopic grasper which can also be used to grasp and manipulate sheet 52 once it is deployed into the body cavity. In alternative embodiments, pushing tool 82 can comprise an elongated rod or other elongated laparoscopic device that is designed or sized to pass through channel 90 of trocar 80.

Once sheet 52 is deployed into the body cavity, surgical instrument 10B/insertion device 12B is removed from trocar 80. Sheet 52 can then be manipulated and positioned laparoscopically in a traditional manner to complete the surgical treatment, such as hernia repair. In one embodiment, slot 32 is sized so that pushing tool 82 can pass out of passage 34 of surgical instrument 10B/insertion device 12B by passing laterally through slot 32. This enables surgical instrument 10B/insertion device 12B to be separated from trocar 80 and pushing tool 82 without having to fully withdraw pushing tool 82 out of trocar 80. This is particularly helpful where pushing tool 82 comprises a laparoscopic grasper or other instrument that is used in the positioning and/or securing of sheet 52 within the abdominal cavity. Thus, in one embodiment, the portion of pushing tool 82 that is disposed within passage 34 of surgical instrument 10B/insertion device 12B has a diameter, such as a maximum diameter, that is smaller than the width of slot 32. However, in alternative embodiments, pushing tool 82 can be sized so that it cannot pass through slot 32. In this embodiment, surgical instrument 10B/insertion device 12B can be separated from pushing tool 82 by either sliding surgical instrument 10B/insertion device 12B over a proximal end of pushing tool 82 while the distal end thereof remains disposed within trocar 80 or by removing pushing tool 82 from trocar 80 and then sliding surgical instrument 10B/insertion device 12B over the distal end of pushing tool 82.

It is appreciated that the surgical instruments/insertion devices and the alternatives and related methods discussed herein have a number of benefits. For example, the insertion devices have a simple and inexpensive design that is easy to manufacture and have no moving parts. Operation of the insertion devices provide a simple and relatively quick approach for both coiling sheet 52 and deploying sheet 52 into the body cavity. As a result, the length of the medical procedure is decreased, thereby decreasing risk to the patient and minimizing cost. The grasping tool 72, pushing tool 82, and trocar 80 can all be tools that are already commonly used in laparoscopic hernia operations. As such, the use of new tools and training is minimal. Furthermore, operation of the insertion devices minimizes risk of tearing of the mesh sheet and damage to other related instruments.

In addition, use of the insertion devices limits or avoids unwanted removal of the trocar from the patient. That is, when complications arise from trying to insert the surgical mesh through a trocar using conventional methods, the surgeon may elect to remove the trocar and advance the surgical mesh directly through the incision in the patient. This is done because the patient's skin can be stretched to enlarge the opening for the mesh. However, repeated removal of the trocar is undesirable in that it results in loss of gas that is pumped into the body cavity as part of the laparoscopic surgery and because it poses a potential risk of damage to organs or other tissue.

Furthermore, use of the insertion devices minimizes the size of trocar needed. That is, to help facilitate insertion of the surgical mesh through a trocar using conventional methods, surgeons may elect to use trocars having a diameter larger than is needed for the operation. Using larger than needed trocars is typically undesirable in that it results in a larger incision being made in the patient which can increase complications such as bleeding, improper healing, and future hernias. Other benefits of the inventive insertion devices and methods of use also exist.

In the previously discussed embodiment, first portion 55 of sheet 52 is secured to proximal end 24 of tube 14B by the surgeon or operator manually holding first portion 55 against proximal end 24. As previously mentioned, however, there are also a plurality of mechanical features or means for securing first portion 55 of sheet 52 to proximal end 24 of tube 14B or any of the other tubes 14 disclosed herein. By way of example and not by limitation, depicted in FIGS. 8A and 8B is another alternative embodiment of a surgical instrument 10C comprising an insertion device 12C. Insertion device 12C comprises a tube 14C having handle 16 projecting therefrom. Like elements between insertion devices 12A, 12B and 12C are identified by like reference characters. Specifically, except as noted below, insertion device 12B and 12C are the same and all of the prior discussion of insertion device 12B, including all discussions of elements, alternatives, materials, sizes, uses and the like also applicable to insertion device 12C. Tube 14C differs from tube 14B in that a notch 102 extends through sidewall 18 at distal end 26 and passes through distal end face 30. Notch 102 is depicted as being elongated and formed on bottom face 38 so as to be disposed opposite of slot 32. This positioning of notch 102 helps to improve visibility of notch 102 through slot 32 during operation, as discussed below. However, in alternative embodiments, notch 102 could be formed on side face 40 or 42. During use, first portion 55 of sheet 52 is passed through slot 32 at distal end 26 and into passage 34, as previously discussed with regard to FIG. 3. However, in contrast to folding terminal section 60 against or around distal end face 30 (FIG. 4), terminal section 60 is now passed into notch 102. Terminal section 60 can be wedged or otherwise frictionally held within notch 102 so as to secure first portion 55 to sheet 52 to distal end 26 of tube 14C. When it is time to deploy coiled sheet 52 from within passage 34, as previously discussed with regard to FIGS. 6 and 7, terminal section 60 can be pulled out of notch 102 so as to be in alignment with passage 34/axis 35. The deployment of coiled sheet 52 then proceeds as previously discussed.

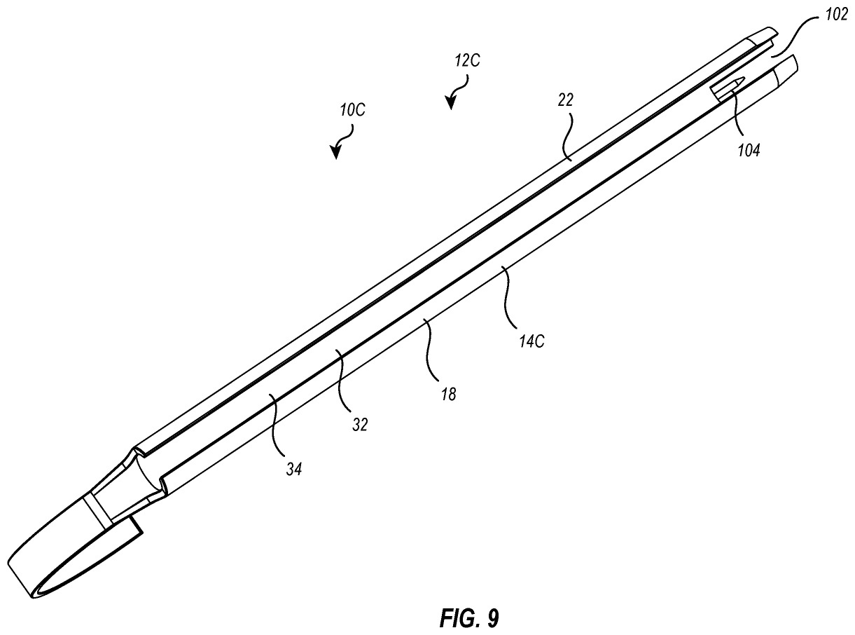

In the embodiment depicted in FIGS. 8A and 8B, notch 102 has a rectangular configuration and typically has a width (maximum or minimum) that is less than a minimum width of slot 32. In alternative embodiments, however, notch 102 can have a variety of other configurations and/or can have elements projecting therein. For example, surgical instrument 10C is again shown in FIG. 9 comprising insertion device 12C. However, in this embodiment, a pointed catch 104 projects from tube 14C/sidewall 18 into notch 102 or into alignment with notch 102. Catch 104 is disposed outside of passage 34 and can project from exterior surface 22, such as from bottom face 38 (FIG. 8B). Catch 104 helps to securely engage terminal section 60 of sheet 52 (FIG. 3) when terminal section 60 is folded into notch 102, as previously discussed. Catch 104 can have a variety of different configurations for engaging terminal section 60.

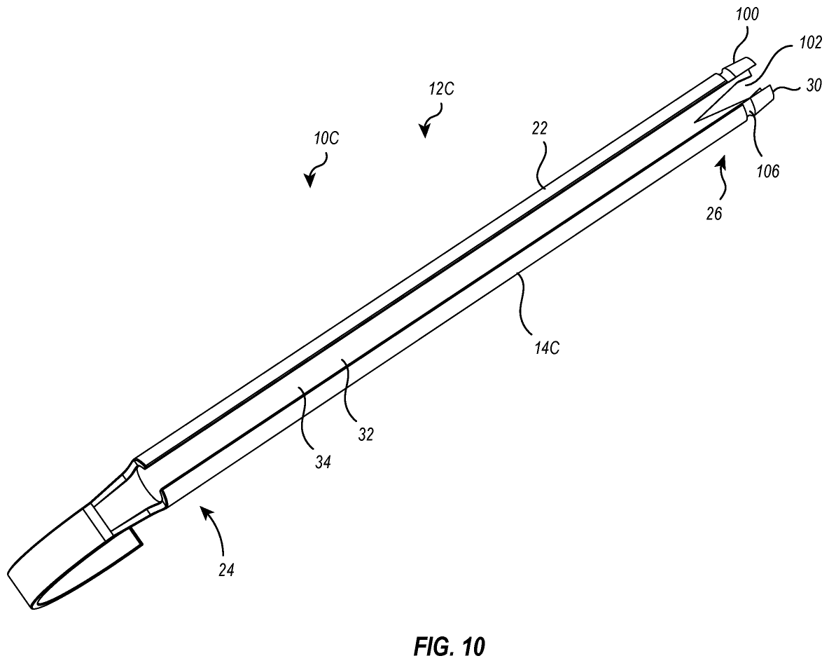

In a further alternative embodiment shown in FIG. 10, surgical instrument 10C is again shown comprising insertion device 12C. However, in this embodiment, notch 102 is now tapered. That is, notch 102 constricts as it extends from distal end face 30 toward proximal end 24. The tapering of notch 102 helps to wedge or otherwise secure terminal section 60 of sheet 52 when terminal section 60 is folded into notch 102, as previously discussed, but still allows easy removal of terminal section 60 from notch 102. In other alternative embodiments, the tapering or shape of notch 102 can have a variety of different configurations.

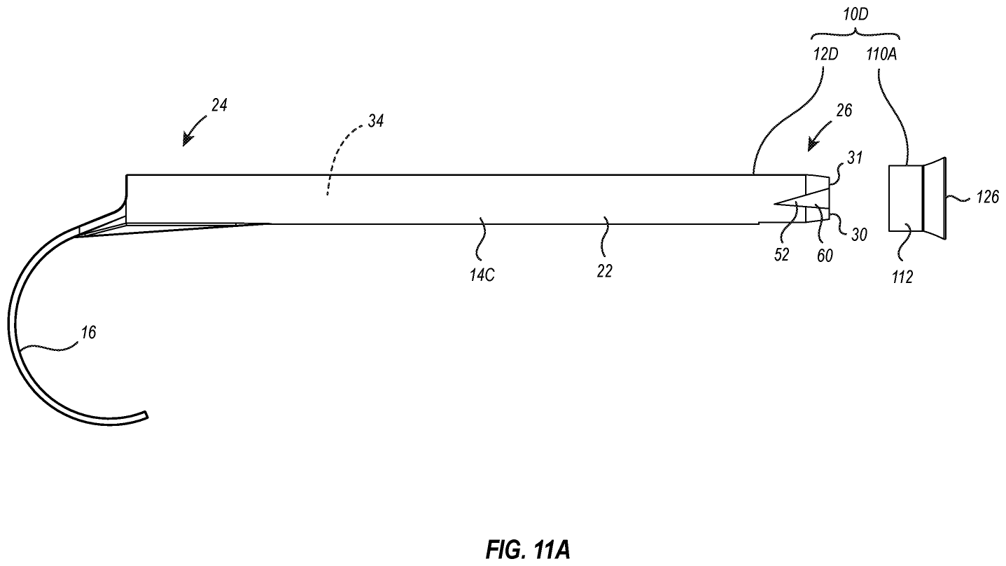

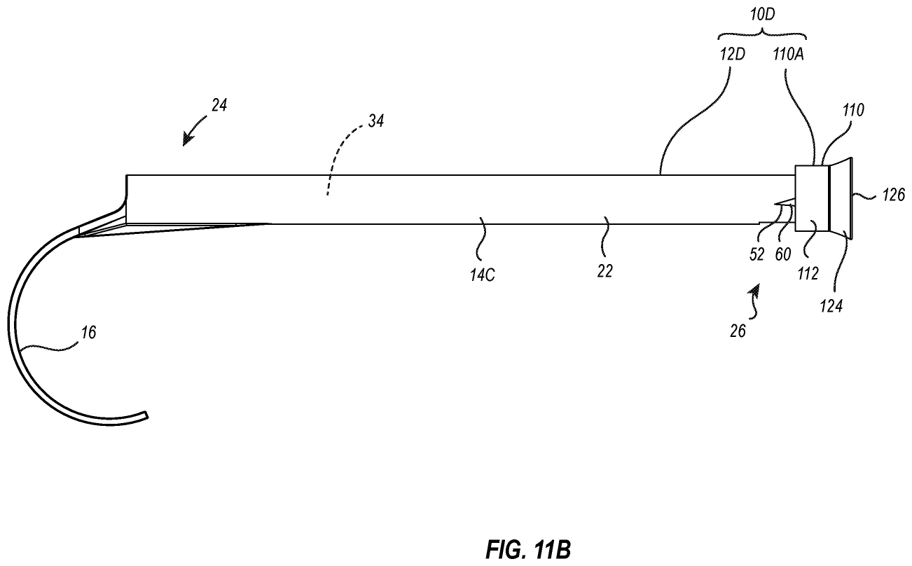

As also depicted in FIG. 10, a groove 106 is recessed into the exterior surface 22 of tube 14C at distal end 26 so as to partially encircle tube 14C. Groove 106 is typically disposed on or at the proximal end of tapered nose 100. Groove 106 can used for removably securing a cap to tube 14C. That is, in contrast to or in combination with the use of notch 102, a cap can be used with insertion device 12B for securing first portion 55 of sheet 52 to distal end 26 of tube 14C. By way of example, depicted in FIGS. 11A and 11B is a surgical instrument 10D which comprises an insertion device 12D and a cap 110A. Insertion device 12D can comprise insertion device 12A, 12B, 12C or any of the other insertion devices disclosed herein. Like element between insertion devices 12C and 12D are identified by like reference characters.

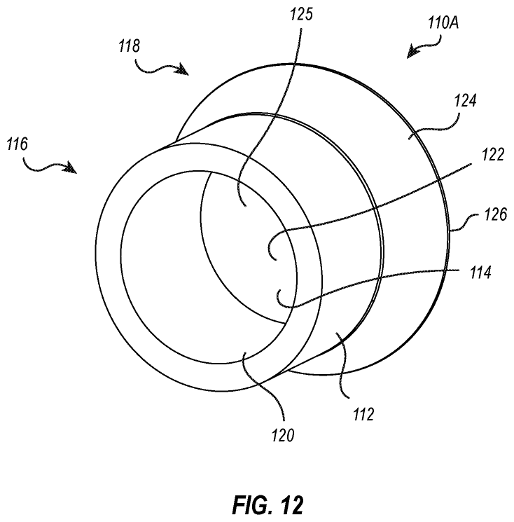

Except as noted below, insertion device 12D can be used in the same method as previously discussed with regard to FIGS. 3-7. In this embodiment, however, cap 110A is configured to removably couple to distal end 26 of tube 14C. More specifically, as depicted in FIG. 12, cap 110A comprises a tubular sleeve 112 at least partially bounding a cavity 114 and having a first end 116 and an opposing second end 118. An opening 120 is formed at first end 116 of sleeve 112 so as to communicate with cavity 114. An end wall 122 is formed at second end 118 of sleeve 112 so as to at least partially bound cavity 114 thereat. End wall 122 has an inside face 125 facing cavity 114 and an opposing outside face 126 (FIG. 11A). Outside face 126 is typically flat so as to make it easy to push cap 110A onto insertion device 12D. Although not required, in the depicted embodiment an optional annular flange 124 encircles and radially outwardly projects from sleeve 112 at second end 118. Flange 124 assists in grasping and handling cap 110A and assists in the attachment and removal from insertion device 12D.

Cap 110A is configured so that distal end 26 of tube 14C can be received within cavity 114 of cap 110A. More specifically, during use, as previously discussed with regard to FIG. 3, the surgeon or operator positions first portion 55 of sheet 52 within distal end 26 of passage 34 so that a terminal section 60 of first portion 55 projects out of passage 34 through distal opening 31. In the current embodiment, the operator can then fold the exposed terminal section 60 laterally, such as around distal end face 30, as shown in FIG. 11A. The surgeon can then advance cap 110A over distal end 26 of tube 14C. Cap 110A is configured so that in this position, terminal section 60 of sheet 52 is wedged between or is otherwise held by frictional engagement between the interior surface of cap 110A and exterior surface 22 of tube 14C. Cap 110A thus securely holds first portion 55 of sheet 52 at distal end 26 of tube 14C. The remaining process of coiling sheet 52 within passage 34, as previously discussed with regard to FIGS. 4 and 5, can then be performed. However, prior to deployment of coiled sheet 52, cap 110A is removed and terminal section 60 straightened so as to be aligned with passage 34 of tube 14C. The deployment of coiled sheet 52 can then occur in the same manner as previously discussed with regard to FIGS. 6 and 7.

It is appreciated that cap 110A can have a variety of alternative configurations. For example, in the depicted embodiment, end wall 122 is solid so as to complete close cavity 114 at second end 118. However, in alternative embodiments, one or more openings could be formed through end wall 122. Tubular sleeve 112 is also shown as being solid. Again, in alternative embodiments, one or more openings could be formed through sleeve 112. In another alternative, end wall 122 could be eliminated so that the cap simply comprises sleeve 112 which can be wedged onto distal end 26 of tube 14C.

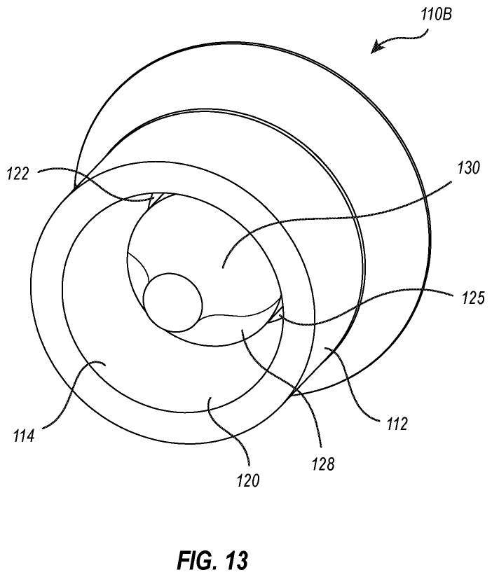

In yet another alternative embodiment depicted in FIG. 13, a cap 110B is shown. Like elements between caps 110A and 110B are identified by like reference characters. All of the above alternative discussed with regard to cap 110A are also applicable to cap 110B and the other caps 110 disclosed herein. Cap 110B has the same configuration and is used in the same manner as cap 110A. The only distinction is that cap 110B also includes a projection 128 centrally projects from inside face 125 of end wall 122 into cavity 114. Projection 128 terminates at an optional rounded nose 130. Rounded nose 130 is typically disposed within cavity 114 of cap 110B but can project outside of sleeve 112. During use, projection 128 is configured to be received within passage 34 of tube 14C at distal end 26 (FIG. 11A) as cap 110B is advanced over distal end 26. Projection 128 can function to wedge or otherwise frictionally engage first portion 55 of sheet 52 between projection 128 and interior surface 88 of tube 14C so as to further assist in securing first portion 55 of sheet 52 to distal end 26.

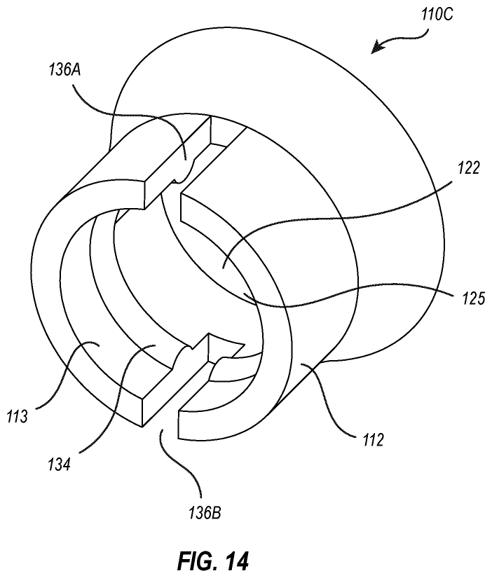

Depicted in FIG. 14 is another alternative embodiment of a cap 110C. Like elements between caps 110A and 110C are identified by like reference characters. Cap 110C has substantially the same configuration as cap 110A and is used in the same manner as cap 110A. Cap 110C differs from cap 110A in that cap 110C includes a ridge 134 radially inwardly projecting from an interior surface 113 of sleeve 112 into cavity 114. In one embodiment, ridge 134 is formed so as to encircle or encircle a portion of cavity, i.e., ridge 134 can be in the form of a ring or partial ring. Ridge 134 is configured to fit within groove 106 (FIG. 10) which can be formed on any of the tubes 14 disclosed therein. More specifically, cap 110C is advanced onto distal end 26 of tube 14C until ridge 134 snap-fits into groove 106 (FIG. 10) so as to make a secure but removable snap-fit connection between cap 110C and tube 14. In one embodiment, slots 136A and 136B (FIG. 14) are formed that pass through sleeve 112 and extend from first end 116 toward second end 118. Slots 136 enable sleeve 112 to more easily radially, outwardly expand as ridge 134 advanced along tapered nose 100 to groove 106 (FIG. 10), thereby providing easier attachment and release of cap 110C to tube 14. However, in other embodiments, a single slot 136 can be used or three or more slots 136 can be used. In still other embodiments, depending on the sizing and material for cap 110C, slots 136 can be eliminated.

In one method of use, cap 110C can be used in the same manner as caps 110A and 110B, previously discussed. In an alternative method of use, terminal section 60 of sheet 52 is passed through one of slots 136 of cap 110C as cap 110C is secured to distal end 26 of tube 14C. Cap 110C would still function to help secure first portion 55 of sheet 52 to distal end 26 of tube 14.

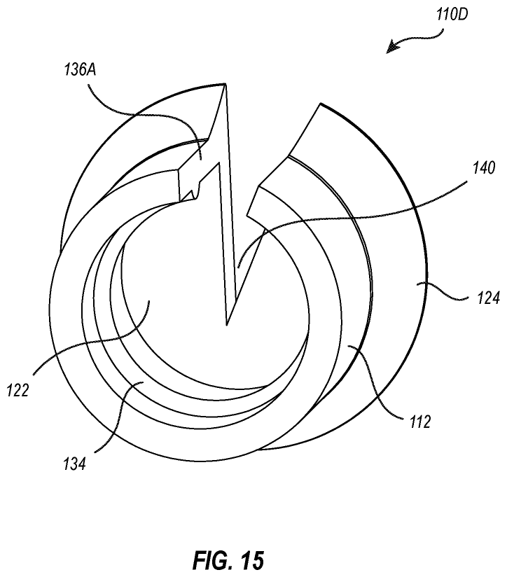

Depicted in FIG. 15 is another alternative embodiment of a cap 110D. Like elements between caps 110C and 110D are identified by like reference characters. Cap 110D differs from cap 110A in that cap 110D only includes a single slot 136A. Cap 110D also differs from cap 110C in that cap 110D includes a notch 140 that extends through optional flange 124 and extends through end wall 122 from an outer perimeter edge thereof to a central portion. In this embodiment, notch 140 inwardly tapers as it extends to the central portion. Notch 140 aligns with and directly communicates with slot 136A.

During use, cap 110D is advanced onto distal end 26 of tube 14C (FIG. 10) so as to produce a snap fit connection therewith, as previously discussed. However, in contrast to securing terminal section 60 of sheet 52 between cap 110D and distal end of tube 14C, terminal section 60 is passed through and secured within notch 140. For example, terminal section 60 can be wedged or otherwise held by frictional engagement within tapered notch 140. As a result, cap 110D secures first portion 55 of sheet 52 to distal end 26 of tube 14B. Again, prior to deployment of coiled sheet 52 from tube 14C, as previously discussed with regard to FIGS. 6 and 8, cap 110D is removed from tube 14C.

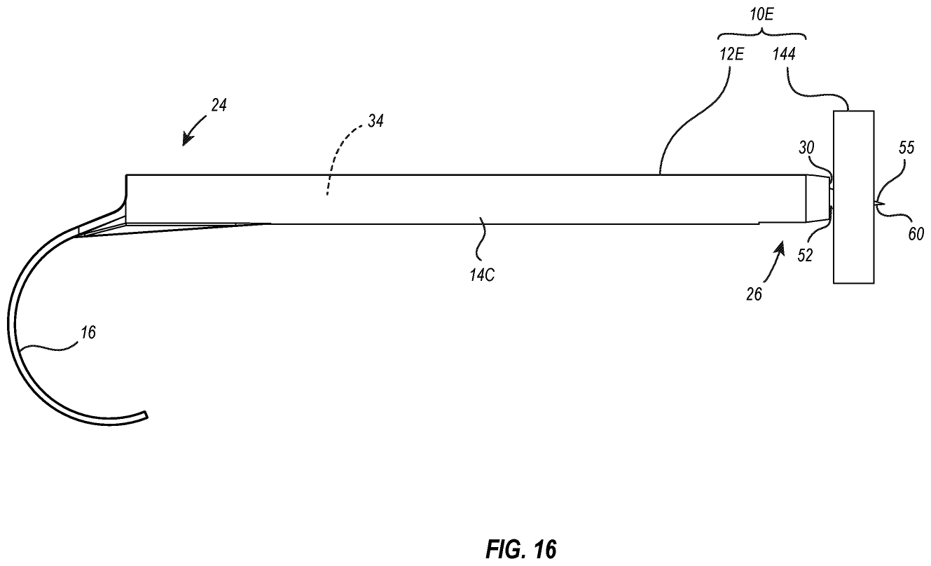

In contrast to using a cap and/or slot on a tube 14/insertion device 12, is appreciated that other mechanisms can also be used for securing first portion 55 of sheet 52 to distal end 26 of a tube 14/insertion device 12. By way of example, depicted in FIG. 16 is a surgical instrument 10E which comprises an insertion device 12E and a clamp 144. Insertion device 12E can comprise insertion device 12A, 12B, 12C, 12D or any of the other insertion devices disclosed herein. Like elements between insertion devices 12B and 12E are identified by like reference characters. Clamp 144 removably clamps onto first portion 55/terminal section 60 of sheet 52 that is exposed distal of distal end face 30 of tube 14C. Clamp 144 is also larger than passage 34 so that clamp 144 cannot be pulled therethrough. As such, as sheet 52 is being coiled within passage 34, as previously discussed, clamp 144 secures first portion 55 of sheet 52 to distal end 26 of tube 14C.

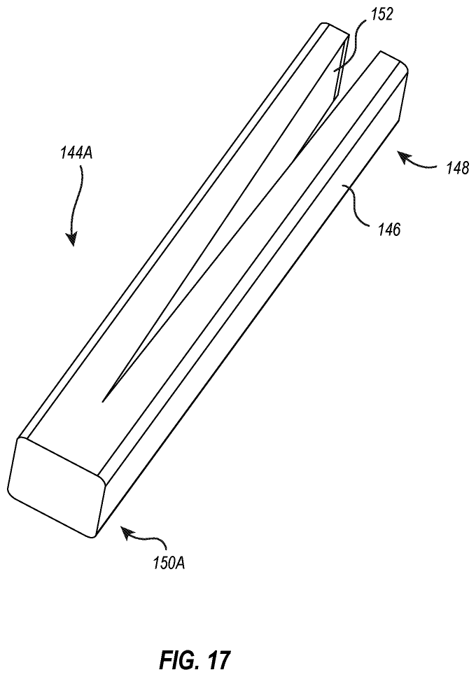

It is appreciated that clamp 144 can have a variety of different configurations. For example, depicted in FIG. 17 is one example of slide clamp 144A that can be used as clamp 144. Slide clamp 144A comprises a body 146, which is typically elongated, and extends from a first end 148 to an opposing second end. A notch 152, which is typically tapered or wedge shaped, passes into body 146 from first end 148 toward second end 150. During use, first portion 55 of sheet 52 projecting from distal end 26 of tube 14C (FIG. 16) can be slid into notch 152 so as to be wedged or otherwise held by frictional engagement therein. When it is desired to deploy coiled sheet 52 from tube 14C, slide clamp 144A is removed and the deployment occurs as previously discussed.

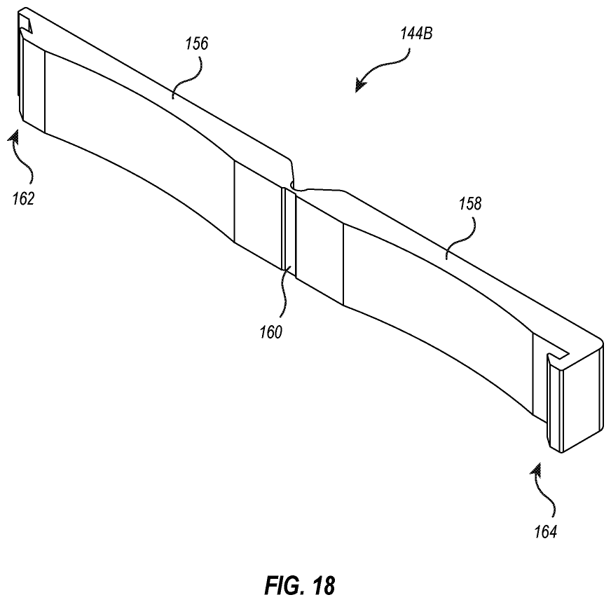

Depicted in FIG. 18 is one example of a hinge clamp 144B that can be used as clamp 144. Hinge clamp 144B comprises a first arm 156, a second arm 158, and a hinge 160 extending therebetween. In one embodiment, hinge 160 can be a living hinge that is integrally formed with arms 156 and 158. Hinge clamp 144B can be closed so that first portion 55 of sheet 52 projecting from distal end 26 of tube 14C (FIG. 16) is clamped and secured between arms 156 and 158. Releasable latch portions 162 and 164 are formed at the free ends of arms 156 and 158, respectively, which can releasably hold hinge clamp 144B in the closed position. Other clamps can also be used.

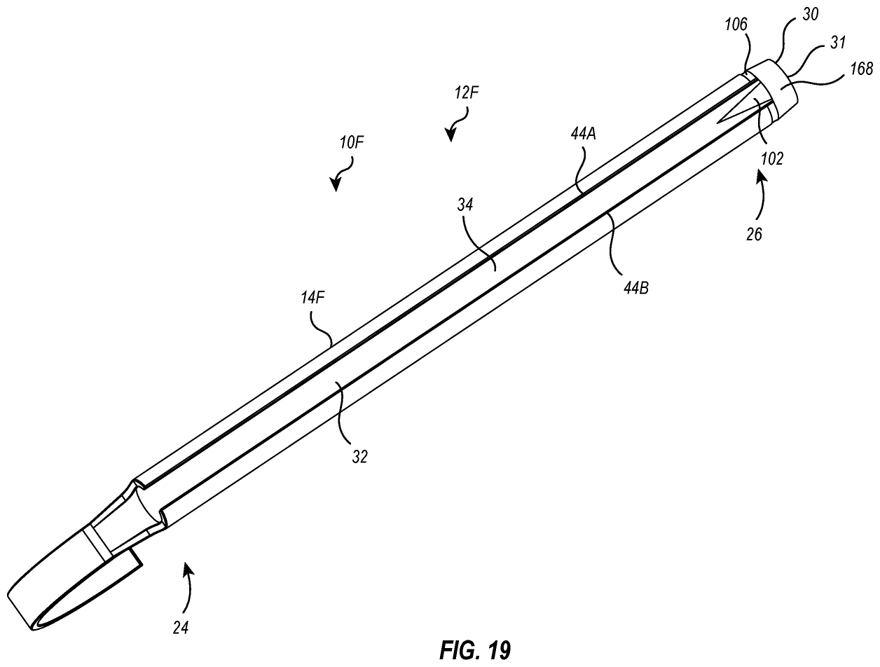

In the prior embodiments of the disclosed insertion devices 12 and tubes 14, slot 32 extends the entire length of tubes 14 between proximal end face 28 and distal end face 30. In alternative embodiments, however, it is also understood that slot 32 need not extend the full length of tubes 14. For example, depicted in FIG. 19 is a surgical instrument 1OF that comprises an insertion device 12F having a tube 14F. Like elements between insertion devices 12C and 12F are identified by like references charters. Insertion devices 12C and 12F are the same expect that for insertion device 12F, slot 32 does not extend the full length of tube 14F to distal end face 30 but rather extends a majority of the length tube 14F. Specifically, slot 32 terminates at distal end 26 at a distance spaced apart from distal end face 30. As such, a sidewall section 168 extends between opposing edges 44A and 44B of sidewall 18 at a location between slot 32 and distal end face 30. Expressed in other terms, in some embodiments distal end face 30 can completely encircle passage 34. Insertion device 12F is used in the same way as the other insertion devices 12, as previously discussed. However, when initially inserting first portion 55 of sheet 52 into passage 34 at distal end 26, first portion 55 needs to be inserted through slot 32 proximal of sidewall section 168 and then either advanced within passage 34 out through distal opening 31 and/or through notch 102. In other embodiments, notch 102 could be eliminated.

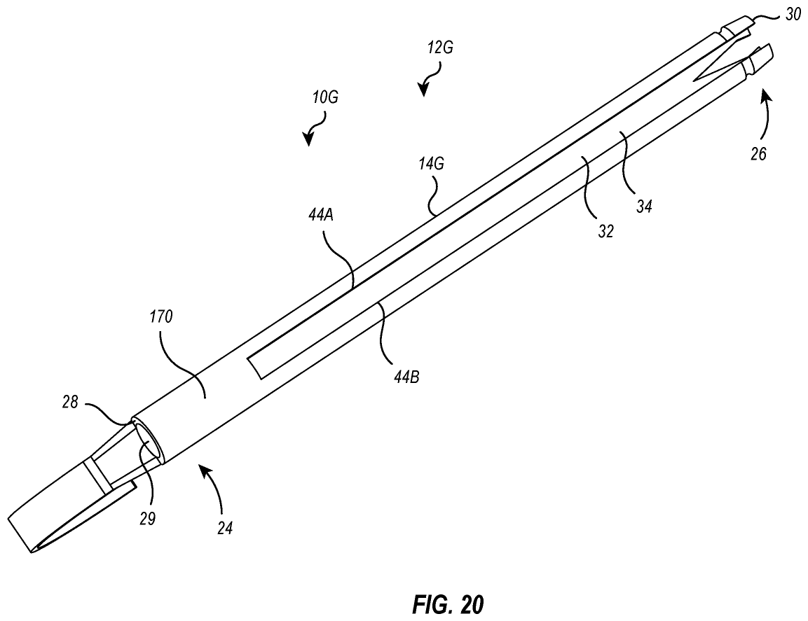

Depicted in FIG. 20 is a further surgical instrument 10G that comprises an insertion device 12G having a tube 14G. Like elements between insertion devices 12F and the other insertion devices disclosed herein are identified by like references charters. Insertion devices 12C and 12G are the same expect that for insertion device 12G, slot 32 again does not extend the full length of tube 14G. Slot 32 does extend through distal end face 30 and extends a majority of the length tube 14G. However, slot 32 does not extend through proximal end face 28. Rather, slot 32 terminates at proximal end 24 at a distance spaced apart from proximal end face 28. As such, a sidewall section 170 extends between opposing edges 44A and 44B of sidewall 18 at a location between slot 32 and proximal end face 28. Expressed in other terms, in some embodiments proximal end face 28 can completely encircle passage 34. Insertion device 12G can be used in the same way as the other insertion devices as previously discussed herein. However, during the coiling process of sheet 52, grasping tool 72 is passed through proximal opening 29 and under sidewall section 170 to grasp and twist second portion 65 that is feed into slot 32 distal of sidewall section 170. However, deployment of the coiled sheet 52 within passage 34 is performed in the same way as previously discussed with regard to FIGS. 6 and 7.

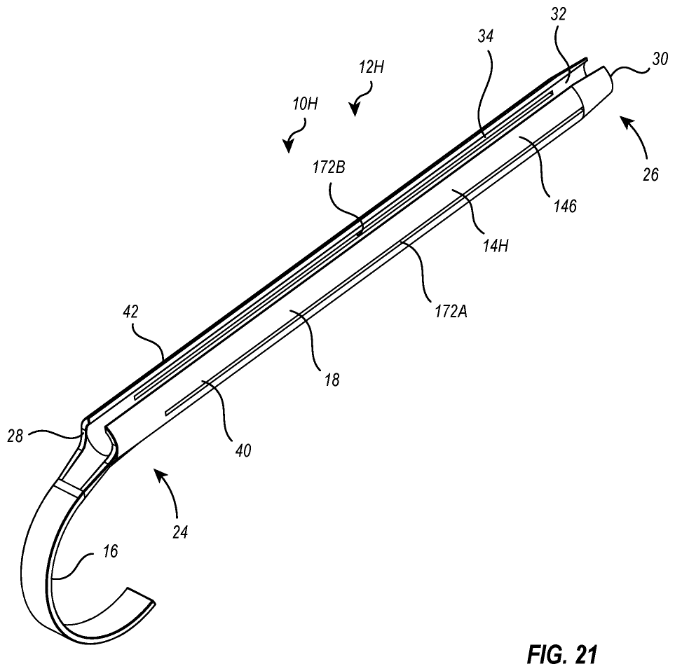

Turning to FIG. 21 is a further surgical instrument 10H that comprises an insertion device 12H having a tube 14H. Like elements between insertion devices 12H and the other insertion devices disclosed herein are identified by like references charters. Insertion devices 12B and 12H are substantially the same. The main distinction between tube 14B and tube 14H is that tube 14H includes elongated secondary slots 172A and 172B that pass through sidewall 18 at side faces 40 and 42, respectively, along the length of tube 14H. Secondary slots 172A and 172B extend between proximal end 24 and distal end 26 but typically do not extend to proximal end face 28 or distal end face 30. Each secondary slot 172A and 172B is sized to receive sheet 52 but typically have a width smaller than the width of slot 32. Secondary slots 172A and 172B typically have a width in a range between 0.5 mm and 3 mm with between 0.5 mm and 2 mm being more common.

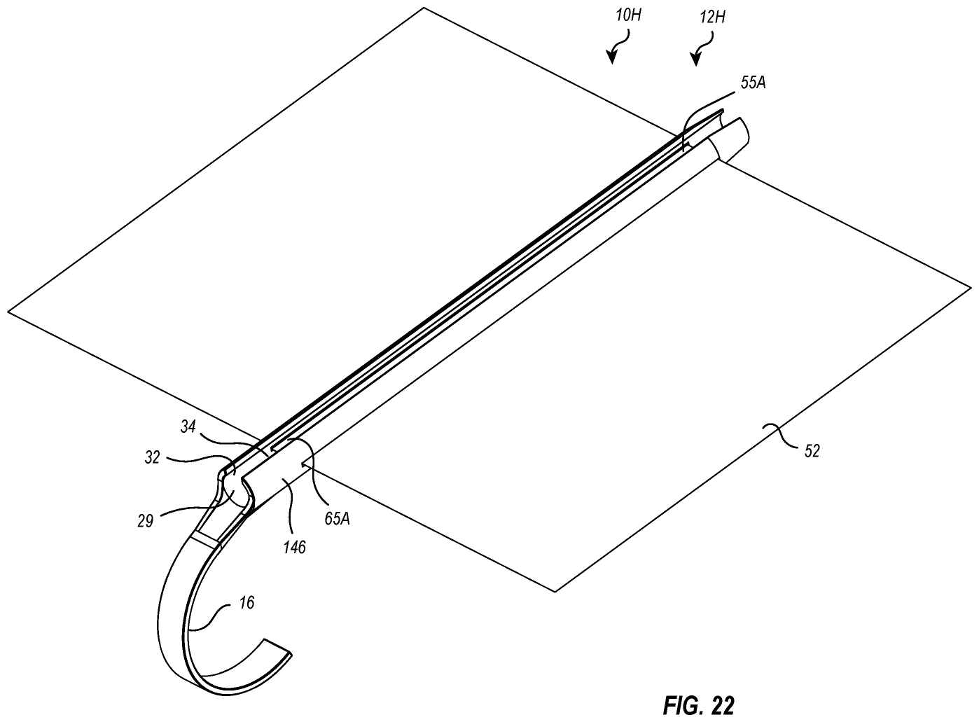

Turning to FIG. 22, during use sheet 52 is passed though secondary slots 172A and 172B so as to span across passage 34. The center of sheet 52 can be disposed within passage 34 but exact centering is not required. By positioning sheet 52 through slots 172A and 172B, a first portion 55A of sheet 52 is secured to distal end 26 of tube 14H. Grasping tool 72 (FIG. 4) is then advanced into passage 34 through proximal opening 29 and used to grasp a second portion 65A of sheet 52 within passage 34. Grasping tool 72 is then manually rotated so as to cause sheet 52 to coil within passage 34. As sheet 52 coils, the exposed opposing ends of sheet 52 are drawn into passage 34. The process of coiling continues until all of sheet 52 is coiled within passage 34. Deployment of sheet 52 is then accomplished using the same method as previously discussed with regard to FIGS. 6 and 7.

It is appreciated that the insertion devices 12 disclosed herein can be provided in a kit that also includes one or more sheets 52. The kit can also include one or more caps 110 and/or grasping tool 72 and/or pushing tool 82, and/or trocar 80. In other embodiments, the kit can include two or more insertions devices 12 of different size or configuration in combination with one or more of the foregoing.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure. For example, present disclosure provides multiple alternatives for the tubes 14, caps 110, notches 102, clamps 144, and other elements. It is appreciated that the present disclosure both invasions and includes that each of the alternatives disclosed herein can be mixed and matched to form a plurality of other alternative embodiments.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A surgical instrument for use in inserting a flexible sheet within a patient, the surgical instrument, comprising:
a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along an entire length of the tube between the proximal end face and the distal end face, the interior surface bounding a passage extending through the tube; and
a handle projecting from the tube at or toward the proximal end.

2. The surgical instrument as recited in claim 1, wherein the slot extending between the proximal end face and the distal end face is linear.

3. The surgical instrument as recited in claim 1, wherein the handle has a U-shaped configuration.

4. The surgical instrument as recited in claim 1, wherein the distal end of the tube is tapered so as to have a frustoconical configuration.

5. The surgical instrument as recited in claim 1, further comprising means for securing the flexible sheet to the distal end of the tube.

6. The surgical instrument as recited in claim 1, further comprising a notch extending through the sidewall at the distal end of the tube and passing through the distal end face.

7. The surgical instrument as recited in claim 6, wherein the notch is disposed opposite the slot.

8. The surgical instrument as recited in claim 1, further comprising a cap removably secured to the distal end of the tube so as to at least partially cover the distal end face.

9. The surgical instrument as recited in claim 8, wherein the cap comprises:
a tubular sleeve at least partially bounding a cavity and having a first end and an opposing second end, opening being formed at the first end of the sleeve so as to communicate with the cavity, the distal end of the tube being disposed within the cavity; and
an end wall formed at the second end of the sleeve so as to at least partially bound the cavity.

10. The surgical instrument as recited in claim 9, further comprising a notch passing through the sleeve so as to communicate with the cavity.

11. A surgical system comprising:
the surgical instrument as recited in claim 1; and
a flexible sheet being removably disposed within the passage of the tube.

12. The surgical system as recited in claim 11, wherein the flexible sheet comprises a surgical mesh.

13. The surgical system as recited in claim 11, wherein the flexible sheet within the passage of the tube is spirally wound.

14. A surgical instrument for use in inserting a flexible sheet within a patient, the surgical instrument, comprising:
a tube having a sidewall with an interior surface and an opposing exterior surface that extend between a proximal end and an opposing distal end, the proximal end terminating at a proximal end face and the distal end terminating at a distal end face, a slot passing through the sidewall of the tube between the interior surface and the exterior surface and extending along at least a portion of a length of the tube between the proximal end face and the distal end face, the interior surface bounding a passage extending through the tube;
a notch extending through the sidewall at the distal end of the tube and passing through the distal end face; and
a handle projecting from the tube at or toward the proximal end.

15. The surgical instrument as recited in claim 14, wherein the notch is tapered.

16. A surgical system comprising:
the surgical instrument as recited in claim 14; and
a flexible sheet, a portion of the flexible sheet being secured within the notch.

17. A method for deploying a sheet of surgical mesh, the method comprising:
securing a first portion of a sheet of surgical mesh to a distal end of a tube of a surgical instrument, the tube having a sidewall with an interior surface and an opposing exterior surface that extend between a proximal end and the opposing distal end, the proximal end terminating at a proximal end face and the distal end terminating at a distal end face, a slot passing through the sidewall of the tube between the interior surface and the exterior surface and extending along at least a portion of a length of the tube between the proximal end face and the distal end face, the interior surface bounding a passage extending through the tube; and
passing a second portion of the sheet through the slot of the tube and into the passage.

18. The method as recited in claim 17, wherein the step of securing the first portion of the sheet comprises securing the sheet within a notch extending through the sidewall at the distal end of the tube and passing through the distal end face.

19. The method as recited in claim 17, wherein the step of securing the first portion of the sheet comprises securing the first portion of the sheet to a cap removably secured to the distal end of the tube so as to at least partially cover the distal end face.

20. The method as recited in claim 17, further comprising after the step of passing a second portion of the sheet through the slot, twisting the sheet so that at least a portion of the sheet is spirally wound within the passage of the tube.
